(19) United States
(12) Patent Application Publication
(10) Pub. No.: US 2010/0150969 A1
(43) Pub. Date: Jun. 17, 2010

(54) IMMUNE RESPONSE

(76) Inventors: Bruce Acres, Strasbourg (FR); Jean-Yves Bonnefoy, Strasbourg (FR)

Correspondence Address: BUCHANAN, INGERSOLL & ROONEY PC, POST OFFICE BOX 1404, ALEXANDRIA, VA, 22313-1404, US

(21) Appl. No.: 12/517,242

(22) PCT Filed: Dec. 4, 2007
(86) PCT No.: PCT/EP07/10527

§ 371 Date: Feb. 16, 2010

(30) Foreign Application Priority Data
Dec. 5, 2006 (EP) 06360056.3

(51) Int. Cl. A61K 39/00; A61P 35/00; A61P 37/04

(52) U.S. Cl. 424/277.1; 424/184.1

(57) ABSTRACT

Immune responses to a targeted antigen are increased in patients for treating human disease, by administering an immunogenic composition, wherein such patients are selected from a patient population of interest.

The present invention relates to the field of immunology and, in particular, to a vaccination procedure for treatment of a patient against diseases caused for example by infection, or cancers. The present invention relates to methods and compositions for improving the immune response raised in vivo by an immunogenic composition, in particular a vaccine.

Traditional vaccination techniques involving the introduction into an animal system of an antigen (e.g. peptides, proteins) which can induce an immune response, and thereby protect said animal against infection for example, have been known for many years. These techniques have further included the development of both live and inactivated vaccines. Live vaccines are typically attenuated non-pathogenic versions of an infectious agent that are capable of priming an immune response directed against a pathogenic version of the infectious agent.

In recent years there have been advances in the development of recombinant vaccines, especially recombinant live vaccines, in which foreign antigens of interest are encoded and expressed from a vector. Amongst them, vectors based on recombinant viruses have shown great promise and play an important role in the development of new vaccines. Many viruses have been investigated for their ability to express proteins from foreign pathogens or tumoral tissue, and to induce specific immunological responses against these antigens in vivo. Generally, these gene-based vaccines can stimulate potent humoral and cellular immune responses and viral vectors might be an effective strategy for both the delivery of antigen-encoding genes and the facilitation and enhancement of antigen presentation. In order to be utilized as a vaccine carrier, the ideal viral vector should be safe and enable efficient presentation of required pathogen-specific antigens to the immune system. Furthermore, the vector system must meet criteria that enable its production on a large-scale basis. Several viral vaccine vectors have thus emerged to date, all of them having relative advantages and limits depending on the proposed application (for a review on recombinant viral vaccines see for example Harrap and Carroll, 2006, Front Biosci., 11, 804-817; Yokoyama et al., 1997, J Vet Med. Sci., 59, 311-322).

Following the observation in the early 1990's that plasmid DNA vectors could directly transfect animal cells in viva, significant research efforts have also been undertaken to develop vaccination techniques based upon the use of DNA plasmids to induce immune response, by direct introduction into animals of DNA which encodes for antigens. Such techniques which are widely referred as DNA vaccination have now been used to elicit protective immune responses in large number of disease models. For a review on DNA vaccines, see Reyes-Sandoval and Ertl, 2001 (Current Molecular Medicine, 1, 217-243).

A general problem in vaccine field however has been the identification of a means of inducing a sufficiently strong immune response in vaccinated individuals to protect against infection and disease.

Therefore there has been for example major effort in recent years, to discover new drug compounds that act by stimulating certain key aspects of the immune system which will serve to increase the immune response induced by vaccines. Most of these compounds, referred as immune response modifiers (IRMs) or adjuvants, appear to act through basic immune system mechanisms via Toll-like receptors (TLRs) to induce various important cytokines biosynthesis (e.g., interferons, interleukins, tumor necrosis factor, etc. see for example Schiller et al., 2006, Exp Dermatol., 15, 331-341). Such compounds have been shown to stimulate a rapid release of certain dendritic cell, monocyte/macrophage-derived cytokines and are also capable of stimulating B cells to secrete antibodies which play an important role in the antiviral and antitumor activities of IRM compounds.

Alternatively, vaccination strategies have been proposed, most of them being based on a prime-boost vaccination regimen. According to these “prime-boost” vaccination protocols, the immune system is first induced by administering to the patient a priming composition and then boosted by administration of a boosting second composition (see for example EP1411974 or US20030191076).

The Applicant has now identified a novel vaccination strategy. According to a first embodiment, the present Invention relates to a method for treating a patient for human disease by administering an immunogenic composition comprising at least one targeted antigen wherein said patient is selected in a patient population composed of patients that have elicited a low to moderate immune response towards an antigen (i.e the prior immune response) and that have not received any prior exogenously supplied pharmaceutical immunogenic composition comprising (i) all or part of the said targeted antigen(s) and/or (ii) at least one recombinant vector encoding the said targeted antigen(s).

The present Invention thus relates to a method for treating a patient for human disease human disease by administering an immunogenic composition comprising at least one targeted antigen, said method comprising the following steps:
selection of patient in a patient population composed of patients that have elicited a low to moderate immune response towards an antigen (i.e the prior immune response) and that have not received any prior exogenously supplied pharmaceutical immunogenic composition comprising (i) all or part of the said targeted antigen(s) and/or (ii) at least one recombinant vector encoding the said targeted antigen(s),
administering to said selected patients the said immunogenic composition.

According to another embodiment, the present Invention relates to a method for raising an immune response to at least one targeted antigen (i.e. the raised immune response) in a patient for treating human disease by administering an immunogenic composition wherein said patient is selected in a patient population composed of patients that have elicited a low to moderate immune response towards an antigen (i.e. the prior immune response) and that have not received any prior exogenously supplied pharmaceutical immunogenic composition comprising (i) all or part of the said targeted antigen(s) and/or (ii) at least one recombinant vector encoding the said targeted antigen(s).

The present Invention thus relates to a method for raising an immune response to at least one targeted antigen (i.e. the raised immune response) in a patient for treating human disease by administering an immunogenic composition, said method comprising the following steps:
selection of patient in a patient population composed of patients that have elicited a low to moderate immune response towards an antigen (i.e the prior immune response) and that have not received any prior exogenously supplied pharmaceutical immunogenic composition comprising (i) all or part of the said targeted antigen(s) and/or (ii) at least one recombinant vector encoding the said targeted antigen(s),
administering to said selected patients the said immunogenic composition.

According to one special embodiment, said “raised immune response” and “low to moderate immune response” in said patient population are directed towards a tumour-specific or -related antigens and/or viral antigen. According to one embodiment, said “raised immune response” and “low to moderate immune response” in said patient population are directed towards distinct antigens. According to one special embodiment, said “raised immune response” and “low to moderate immune response” in said patient population are both directed towards MUC1 antigen. According to another special embodiment, said “raised immune response” and “low to moderate immune response” in said patient population are both T cell immune response, and preferably CD8+ (Cytotoxic T Lymphocytes) immune response.

As used herein throughout the entire application, the terms “a” and “an” are used in the sense that they mean “at least one”, “at least a first”, “one or more” or “a plurality” of the referenced compounds or steps, unless the context dictates otherwise. For example, the term “a cell” includes a plurality of cells including a mixture thereof. More specifically, “at least one” and “one or more” means a number which is one or greater than one, with a special preference for one, two or three.

The term “and/or” wherever used herein includes the meaning of “and”, “or” and “all or any other combination of the elements connected by said term”.

The term “about” or “approximately” as used herein means within 20%, preferably within 10%, and more preferably within 5%.

The term “patient” refers to a vertebrate, particularly a member of the mammalian species and includes, but is not limited to, domestic animals, sport animals, primates including humans.

As used herein, the term “treatment” or “treating” encompasses prophylaxis and/or therapy. Accordingly the immunogenic combinations or methods of the present invention are not limited to therapeutic applications and can be used in prophylaxis ones. “Prophylaxis” is not limited to preventing immediate diseases (e.g. infectious diseases), it further encompasses prevention of long term consequences of these infections such as cirrhosis or cancer.

An “effective amount” or a “sufficient amount” of an active compound is an amount sufficient to effect beneficial or desired results, including clinical results. An effective amount can be administered in one or more administrations. A “therapeutically effective amount” is an amount to effect beneficial clinical results, including, but not limited to, alleviation of one or more symptoms associated with viral infection as well as prevention of disease (e.g. prevention of one or more symptoms of infection).

According to another embodiment, the present Invention relates to a method for raising an immune response to a targeted antigen (i.e. the raised immune response) in a patient for treating human disease by administering an immunogenic composition wherein said patient is selected in a patient population composed of patients that have elicited a low to moderate immune response towards the said targeted antigen (i.e. the prior immune response) and that have not received any prior exogenously supplied pharmaceutical immunogenic composition comprising (i) all or part of the said targeted antigen and/or (ii) at least one recombinant vector encoding the said targeted antigen.

The present Invention thus relates to a method for raising an immune response to a targeted antigen (i.e. the raised immune response) in a patient for treating human disease by administering an immunogenic composition, said method comprising the following steps:
selection of patient in a patient population composed of patients that have elicited a low to moderate immune response towards to the said targeted antigen (i.e the prior immune response) and that have not received any prior exogenously supplied pharmaceutical immunogenic composition comprising (i) all or part of the said targeted antigen(s) and/or (ii) at least one recombinant vector encoding the said targeted antigen(s),
administering to said selected patients the said immunogenic composition.

As used herein, the terms “immunogenic composition” “vaccine composition”, “vaccine” or similar terms can be used interchangeably and mean an agent suitable for stimulating/inducing/increasing a subject's immune system to ameliorate a current condition or to protect against or to reduce present or future harm or infections (including viral, bacterial, parasitic infections), e.g., reduced tumour cell proliferation or survival, reduced pathogen replication or spread in a subject or a detectably reduced unwanted symptom(s) associated with a condition, expend patient survival rate. Said immunogenic composition can contain (i) all or part of at least one targeted antigen and/or (ii) at least one recombinant vector expressing in viva all or part of at least one heterologous nucleotide sequence, especially an heterologous nucleotide sequence encoding all or part of at least one targeted antigen. According to an alternate embodiment, the immunogenic composition of the Invention comprises (iii) at least one immune response modifier, alone or in combination with (i) and/or (ii). Examples of such immune response modifiers (IRMs), include the CpG oligonucleotides (see U.S. Pat. No. 6,194,388; US2006094683; WO 2004039829 for example), lipopolysaccharides, polyinosic:polycytidylic acid complexes (Kadowaki, et al., 2001, J. Immunol. 166, 2291-2295), and polypeptides and proteins known to induce cytokine production from dendritic cells and/or monocyte/macrophages. Other examples of such immune response modifiers (IRMs) are small organic molecule such as imidazoquinolinamines, imidazopyridine amines, 6,7-fused cycloalkylimidazopyridine amines, imidazonaphthyridine amines, oxazoloquinoline amines, thiazoloquinoline amines and 1,2-bridged imidazoquinoline amines (see for example U.S. Pat. No. 4,689,338; U.S. Pat. No. 5,389,640; U.S. Pat. No. 6,110,929; and U.S. Pat. No. 6,331,539).

As used herein, the term “antigen” or “targeted antigen” refers to any substance, including complex antigen (e.g. tumour cells, virus infected cells, etc. . . . ), that is capable of being the target of an immune response. An antigen may be the target of, for example, a cell-mediated and/or humoral immune response raised by a patient. The term “antigen” or “targeted antigen” encompasses for example all or part of viral antigens, tumour-specific or -related antigens, bacterial antigens, parasitic antigens, allergens and the like:
Viral antigens include for example antigens from hepatitis viruses A, B, C, D and E, HIV, herpes viruses, cytomegalovirus, varicella zoster, papilloma viruses, Epstein Barr virus, influenza viruses, para-influenza viruses, adenoviruses, coxsakie viruses, picorna viruses, rotaviruses, respiratory syncytial viruses, pox viruses, rhinoviruses, rubella virus, papovirus, mumps virus, measles virus; some non-limiting examples of known viral antigens include the following: antigens derived from HIV-1 such as tat, nef, gp120 or gp160, gp40, p24, gag, env, vif, vpr, vpu, rev or part and/or combinations thereof; antigens derived from human herpes viruses such as gH, gL gM gB gC gK gE or gD or part and/or combinations thereof or Immediate Early protein such as ICP27, ICP47, ICP4, ICP36 from HSV1 or HSV2; antigens derived from cytomegalovirus, especially human cytomegalovirus such as gB or derivatives thereof; antigens derived from Epstein Barr virus such as gp350 or derivatives thereof; antigens derived from Varicella Zoster Virus such as gpl, 11, 111 and IE63; antigens derived from a hepatitis virus such as hepatitis B, hepatitis C or hepatitis E virus antigen (e.g. env protein E1 or E2, core protein, NS2, NS3, NS4a, NS4b, NS5a, NS5b, p7, or part and/or combinations thereof of HCV); antigens derived from human papilloma viruses (for example HPV6, 11, 16, 18, e.g. L1, L2, E1, E2, E3, E4, E5, E6, E7, or part and/or combinations thereof); antigens derived from other viral pathogens, such as Respiratory Syncytial virus (e.g. F and G proteins or derivatives thereof), parainfluenza virus, measles virus, mumps virus, flaviviruses (e.g. Yellow Fever Virus, Dengue Virus, Tick-borne encephalitis virus, Japanese Encephalitis Virus) or Influenza virus cells (e.g. HA, NP, NA, or M proteins, or part and/or combinations thereof);
tumor-specific or -related antigens include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, liver cancer, vulval cancer, thyroid cancer, hepatic carcinoma and various types of head and neck cancer, renal cancer, malignant melanoma, laryngeal cancer, prostate cancer. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Some non-limiting examples of tumor-specific or -related antigens include MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MACE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUG family (e.g. MUC-1), HER2/neu, p21ras, RCAS1, alpha-fetoprotein, E-cadherin, alpha-catenin, beta-catenin and gamma-catenin, p120ctn, gp100.sup.Pme1117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2;
bacterial antigens includes for example antigens from Mycobacteria causing TB and leprosy, pneumocci, aerobic gram negative bacilli, mycoplasma, staphyloccocal infections, streptococcal infections, salmonellae, chlamydiae, neisseriae;
other antigens includes for example antigens from malaria, leishmaniasis, trypanosomiasis, toxoplasmosis, schistosomiasis, filariasis;
allergens refer to a substance that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

According to one special embodiment, said targeted antigen is encoded by an heterologous nucleotide sequence and is expressed in vivo by a recombinant vector. In a particularly preferred embodiment the heterologous nucleotide sequence of the present invention, encodes one or more of all or part of the following targeted antigens HBV-PreS1 PreS2 and Surface env proteins, core and polHIV-gp120 gp40, gp160, p24, gag, pol, env, vif, vpr, vpu, tat, rev, nef; HPV-E1, E2, E3, E4, E5, E6, E7, E8, L1, L2 (see for example WO 90/10459, WO 98/04705, WO 99/03885); HCV env protein E1 or E2, core protein, NS2, NS3, NS4a, NS4b, NS5a, NS5b, p7 (see for example WO2004111082, WO2005051420); Muc-1 (see for example U.S. Pat. No. 5,861,381; U.S. Pat. No. 6,054,438; WO98/04727; WO98/37095).

According to variants of the invention, the immunogenic composition contains at least two targeted antigens, or an heterologous nucleotide sequence encoding at least two targeted antigens, or at least two heterologous nucleotide sequences encoding at least two targeted antigens, or any combination thereof.

According to another special embodiment, said heterologous nucleotide sequence of the present invention, encodes all or part of HPV antigen(s) selected in the group consisting of E6 early coding region of HPV, E7 early coding region of HPV and derivates or combination thereof.

The HPV antigen encoded by the recombinant vector according to the invention is selected in the group consisting of an HPV E6 polypeptide, an HPV E7 polypeptide or both an HPV E6 polypeptide and an HPV E7 polypeptide. The present invention encompasses the use of any HPV E6 polypeptide which binding to p53 is altered or at least significantly reduced and/or the use of any HPV E7 polypeptide which binding to Rb is altered or at least significantly reduced (Munger et al., 1989, EMBO J. 8, 4099-4105; Crook et al., 1991, Cell 67, 547-556; Heck et al., 1992, Proc. Natl. Acad. Sci. USA 89, 4442-4446; Phelps et al., 1992, J. Virol. 66, 2148-2427). A non-oncogenic HPV-16 E6 variant which is suitable for the purpose of the present invention is deleted of one or more amino acid residues located from approximately position 118 to approximately position 122 (+1 representing the first methionine residue of the native HPV-16 E6 polypeptide), with a special preference for the complete deletion of residues 118 to 122 (CPEEK). A non-oncogenic HPV-16 E7 variant which is suitable for the purpose of the present invention is deleted of one or more amino acid residues located from approximately position 21 to approximately position 26 (+1 representing the first amino acid of the native HPV-16 E7 polypeptide, with a special preference for the complete deletion of residues 21 to 26 (DLYCYE). According to a preferred embodiment, the one or more HPV-16 early polypeptide(s) in use in the invention is/are further modified so as to improve MHC class I and/or MHC class II presentation, and/or to stimulate anti-HPV immunity. HPV E6 and E7 polypeptides are nuclear proteins and it has been previously shown that membrane presentation permits to improve their therapeutic efficacy (see for example WO99/03885). Thus, it may be advisable to modify at least one of the HPV early polypeptide(s) so as to be anchored to the cell membrane. Membrane anchorage can be easily achieved by incorporating in the HPV early polypeptide a membrane-anchoring sequence and if the native polypeptide lacks it a secretory sequence (i.e. a signal peptide). Membrane-anchoring and secretory sequences are known in the art. Briefly, secretory sequences are present at the N-terminus of the membrane presented or secreted polypeptides and initiate their passage into the endoplasmic reticulum (ER). They usually comprise 15 to 35 essentially hydrophobic amino acids which are then removed by a specific ER-located endopeptidase to give the mature polypeptide. Membrane-anchoring sequences are usually highly hydrophobic in nature and serves to anchor the polypeptides in the cell membrane (see for example Branden and Tooze, 1991, in Introduction to Protein Structure p. 202-214, NY Garland).

The choice of the membrane-anchoring and secretory sequences which can be used in the context of the present invention is vast. They may be obtained from any membrane-anchored and/or secreted polypeptide comprising it (e.g. cellular or viral polypeptides) such as the rabies glycoprotein, of the HIV virus envelope glycoprotein or of the measles virus F protein or may be synthetic. The membrane anchoring and/or secretory sequences inserted in each of the early HPV-16 polypeptides used according to the invention may have a common or different origin. The preferred site of insertion of the secretory sequence is the N-terminus downstream of the codon for initiation of translation and that of the membrane-anchoring sequence is the C-terminus, for example immediately upstream of the stop codon.

The HPV E6 polypeptide in use in the present invention is preferably modified by insertion of the secretory and membrane-anchoring signals of the measles F protein. Optionally or in combination, the HPV E7 polypeptide in use in the present invention is preferably modified by insertion of the secretory and membrane-anchoring signals of the rabies glycoprotein.

The therapeutic efficacy of the recombinant vector can also be improved by using one or more nucleic acid encoding immunopotentiator polypeptide(s). For example, it may be advantageous to link the HPV early polypeptide(s) to a polypeptide such as calreticulin (Cheng et al., 2001, J. Clin. Invest. 108, 669-678), Mycobacterium tuberculosis heat shock protein 70 (HSP70) (Chen et al., 2000, Cancer Res. 60, 1035-1042), ubiquitin (Rodriguez et al., 1997, J. Virol. 71, 8497-8503) or the translocation domain of a bacterial toxin such as Pseudomonas aeruginosa exotoxin A (ETA(dIII)) (Hung et al., 2001 Cancer Res. 61, 3698-3703).

According to another and preferred embodiment, the recombinant vector according to the invention comprises a nucleic acid encoding one or more early polypeptide(s) as above defined, and more particularly HPV-16 and/or HPV-18 early E6 and/or E7 polypeptides.

According to another special embodiment, said heterologous nucleotide sequence of the present invention, encodes all or part of MUC 1 antigen or derivates thereof.

According to another special embodiment, said heterologous nucleotide sequence of the present invention, encodes one or more of all or part of the followings: HCV env protein E1 or E2, core protein, NS2, NS3, NS4a, NS4b, NS5a, NS5b, p7 or derivates thereof. According to another special embodiment, said heterologous nucleotide sequence of the present invention, encodes one or more fusion protein wherein the configuration is not native in the sense that at least one of the NS polypeptides appears in an order which is distinct from that of the native configuration. Thus, if the fusion protein comprises a NS3 polypeptide, a NS4A polypeptide and a NS5B polypeptide, the native configuration would be NS3-NS4A-NS5B with NS3 at the N-terminus and NS5B at the C-terminus. In contrast, a non-native configuration can be NS5B-NS3-NS4A, NS5B-NS4A-NS3, NS4A-NS3-NS5B, NS4A-NS5B-NS3 or NS3-NS5B-NS4A. In particular, the fusion protein according to the invention comprises at least one of the followings:
A NS4A polypeptide fused directly or through a linker to the N-terminus of a NS3 polypeptide;
A NS3 polypeptide fused directly or through a linker to the N-terminus of a NS5B polypeptide;
A NS4B polypeptide fused directly or through a linker to the N-terminus of a NS5B polypeptide;
A NS4A polypeptide fused directly or through a linker to the N-terminus of a NS3 polypeptide which is fused directly or through a linker to the N-terminus of a NS4B polypeptide; and/or
A NS3 polypeptide fused directly or through a linker to the N-terminus of a NS4B polypeptide which is fused directly or through a linker to the N-terminus of a NS5B polypeptide.

In such specific portions of the fusion protein of the invention, each of the NS polypeptides can be independently native or modified. For example, the NS4A polypeptide included in the NS4A-NS3 portion can be native whereas the NS3 polypeptide comprises at least one of the modifications described below.

If needed, the nucleic acid molecule in use in the invention may be optimized for providing high level expression of the targeted antigen (e.g. HPV early polypeptide(s)) in a particular host cell or organism, e.g. a human host cell or organism. Typically, codon optimisation is performed by replacing one or more “native” (e.g. HPV) codon corresponding to a codon infrequently used in the mammalian host cell by one or more codon encoding the same amino acid which is more frequently used. This can be achieved by conventional mutagenesis or by chemical synthetic techniques (e.g. resulting in a synthetic nucleic acid). It is not necessary to replace all native codons corresponding to infrequently used codons since increased expression can be achieved even with partial replacement. Moreover, some deviations from strict adherence to optimised codon usage may be made to accommodate the introduction of restriction site(s).

As used herein, the term “recombinant vector” refers to viral as well as non viral vectors, including extrachromosomal (e.g. episome), multicopy and integrating vectors (i.e. for being incorporated into the host chromosomes). Particularly important in the context of the invention are vectors for use in gene therapy (i.e. which are capable of delivering the nucleic acid to a host organism) as well as expression vectors for use in various expression systems. Suitable non viral vectors include plasmids such as pREP4, pCEP4 (Invitrogene), pCI (Promega), pCDM8 (Seed, 1987, Nature 329, 840), pVAX and pgWiz (Gene Therapy System Inc; Himoudi et al., 2002, J. Viral. 76, 12735-12746). Suitable viral vectors may be derived from a variety of different viruses (e.g. retrovirus, adenovirus, AAV, poxvirus, herpes virus, measle virus, foamy virus and the like). As used herein, the term “viral vector” encompasses vector DNA/RNA as well as viral particles generated thereof. Viral vectors can be replication-competent, or can be genetically disabled so as to be replication-defective or replication-impaired. The term “replication-competent” as used herein encompasses replication-selective and conditionally-replicative viral vectors which are engineered to replicate better or selectively in specific host cells (e.g. tumoral cells).

In one aspect, the recombinant vector in use in the invention is a recombinant adenoviral vector (for a review, see “Adenoviral vectors for gene therapy”, 2002, Ed D. Curiel and J. Douglas, Academic Press). It can be derived from a variety of human or animal sources and any serotype can be employed from the adenovirus serotypes 1 through 51. Particularly preferred are human adenoviruses 2 (Ad2), 5 (Ad5), 6 (Ad6), 11 (Ad11), 24 (Ad24) and 35 (Ad35). Such adenovirus are available from the American Type Culture Collection (ATCC, Rockville, Md.), and have been the subject of numerous publications describing their sequence, organization and methods of producing, allowing the artisan to apply them (see for example U.S. Pat. No. 6,133,028; U.S. Pat. No. 6,110,735; WO 02/40665; WO 00/50573; EP 1016711; Vogels et al., 2003, J. Virol. 77, 8263-8271).

The adenoviral vector in use in the present invention can be replication-competent. Numerous examples of replication-competent adenoviral vectors are readily available to those skill in the art (see, for example, Hernandez-Alcoceba et al., 2000, Human Gene Ther. 11, 2009-2024; Nemunaitis et al., 2001, Gene Ther. 8, 746-759; Alemany et al., 2000, Nature Biotechnology 18, 723-727). For example, they can be engineered from a wild-type adenovirus genome by deletion in the E1A CR2 domain (see for example WO00/24408) and/or by replacement of the native E1 and/or E4 promoters with tissue, tumor or cell status-specific promoters (see for example U.S. Pat. No. 5,998,205, WO99/25860, U.S. Pat. No. 5,698,443, WO00/46355, WO00/15820 and WO01/36650).

Alternatively, the adenoviral vector in use in the invention is replication-defective (see for example WO94/28152; Lusky et al., 1998, J. Virol 72, 2022-2032). Preferred replication-defective adenoviral vectors are E1-defective (see for example U.S. Pat. No. 6,136,594 and U.S. Pat. No. 6,013,638), with an E1 deletion extending from approximately positions 459 to 3328 or from approximately positions 459 to 3510 (by reference to the sequence of the human adenovirus type 5 disclosed in the GeneBank under the accession number M 73260 and in Chroboczek et al., 1992, Virol. 186, 280-285). The cloning capacity can further be improved by deleting additional portion(s) of the adenoviral genome (all or part of the non essential E3 region or of other essential E2, E4 regions). Insertion of a nucleic acid in any location of the adenoviral vector can be performed through homologous recombination as described in Chartier et al. (1996, J. Viral. 70, 4805-4810). For example, the nucleic acid encoding the HPV-16 E6 polypeptide can be inserted in replacement of the E1 region and the nucleic acid encoding the HPV-16 E7 polypeptide in replacement of the E3 region or vice versa.

In another and preferred aspect, the vector in use in the invention is a poxviral vector (see for example Cox et al. in “Viruses in Human Gene Therapy” Ed J. M. Hos, Carolina Academic Press). According to another preferred embodiment it is selected in the group consisting of vaccinia virus, suitable vaccinia viruses include without limitation the Copenhagen strain (Goebel et al., 1990, Viral. 179, 247-266 and 517-563; Johnson et al., 1993, Viral. 196, 381-401), the Wyeth strain and the highly attenuated attenuated virus derived thereof including MVA (for review see Mayr, A., et al., 1975, Infection 3, 6-14) and derivates thereof (such as MVA vaccinia strain 575 (ECACC V00120707—U.S. Pat. No. 6,913,752), NYVAC (see WO 92/15672—Tartaglia et al., 1992, Virology, 188, 217-232). Determination of the complete sequence of the MVA genome and comparison with the Copenhagen VV genome has allowed the precise identification of the seven deletions (I to VII) which occurred in the MVA genome (Antoine et al., 1998, Virology 244, 365-396), any of which can be used to insert the antigen-encoding nucleic acid. The vector may also be obtained from any other member of the poxyiridae, in particular fowlpox (e.g. TROVAC, see Paoletti et al, 1995, Dev Biol Stand., 84, 159-163); canarypox (e.g. ALVAC, WO 95/27780, Paoletti et al, 1995, Dev Biol Stand., 84, 159-163); pigeonpox; swinepox and the like. By way of example, persons skilled in the art may refer to WO 92 15672 (incorporated by reference) which describes the production of expression vectors based on poxviruses capable of expressing such heterologous nucleotide sequence, especially nucleotide sequence encoding antigen.

The basic technique for inserting the nucleic acid and associated regulatory elements required for expression in a poxviral genome is described in numerous documents accessible to the man skilled in the art (Paul et al., 2002, Cancer gene Ther. 9, 470-477; Piccini et al., 1987, Methods of Enzymology 153, 545-563; U.S. Pat. No. 4,769,330; U.S. Pat. No. 4,772,848; U.S. Pat. No. 4,603,112; U.S. Pat. No. 5,100,587 and U.S. Pat. No. 5,179,993). Usually, one proceed through homologous recombination between overlapping sequences (i.e. desired insertion site) present both in the viral genome and a plasmid carrying the nucleic acid to insert.

The nucleic acid encoding the antigen of the Invention is preferably inserted in a nonessential locus of the poxviral genome, in order that the recombinant poxvirus remains viable and infectious. Nonessential regions are non-coding intergenic regions or any gene for which inactivation or deletion does not significantly impair viral growth, replication or infection. One may also envisage insertion in an essential viral locus provided that the defective function is supplied in trans during production of viral particles, for example by using an helper cell line carrying the complementing sequences corresponding to those deleted in the poxviral genome.

When using the Copenhagen vaccinia virus, the antigen-encoding nucleic acid is preferably inserted in the thymidine kinase gene (tk) (Hruby et al., 1983, Proc. Natl. Acad. Sci. USA 80, 3411-3415; Weir et al., 1983, J. Viral. 46, 530-537). However, other insertion sites are also appropriate, e.g. in the hemagglutinin gene (Guo et al., 1989, J. Virol. 63, 4189-4198), in the K1L locus, in the u gene (Zhou et al., 1990, J. Gen. Virol, 71, 2185-2190) or at the left end of the vaccinia virus genome where a variety of spontaneous or engineered deletions have been reported in the literature (Altenburger et al., 1989, Archives Viral. 105, 15-27; Moss et al. 1981, J. Virol. 40, 387-395; Panicali et al., 1981, J. Viral. 37, 1000-1010; Perkus et al, 1989, J. Virol. 63, 3829-3836; Perkus et al, 1990, Virol. 179, 276-286; Perkus et al, 1991, Virol. 180, 406-410).

When using MVA, the antigen-encoding nucleic acid can be inserted in anyone of the identified deletions I to VII as well as in the D4R locus, but insertion in deletion II or III is preferred (Meyer et al., 1991, J. Gen. Virol. 72, 1031-1038; Sutter et al., 1994, Vaccine 12, 1032-1040).

When using fowlpox virus, although insertion within the thymidine kinase gene may be considered, the antigen-encoding nucleic acid is preferably introduced in the intergenic region situated between ORFs 7 and 9 (see for example EP 314 569 and U.S. Pat. No. 5,180,675).

According to one special embodiment, said recombinant vector is a recombinant plasmid DNA or a recombinant viral vector.

According to another special embodiment, said recombinant viral vector is a recombinant adenoviral vector.

According to another special embodiment, said recombinant viral vector is a recombinant vaccinia vector.

According to another special embodiment, said recombinant vaccinia vector is a recombinant MVA vector.

Preferably, the antigen-encoding nucleic acid in use in the invention is in a form suitable for its expression in a host cell or organism, which means that the nucleic acid sequence encoding the antigen are placed under the control of one or more regulatory sequences necessary for its expression in the host cell or organism. As used herein, the term “regulatory sequence” refers to any sequence that allows, contributes or modulates the expression of a nucleic acid in a given host cell, including replication, duplication, transcription, splicing, translation, stability and/or transport of the nucleic acid or one of its derivative (i.e. mRNA) into the host cell. It will be appreciated by those skilled in the art that the choice of the regulatory sequences can depend on factors such as the host cell, the vector and the level of expression desired. The nucleic acid encoding the antigen is operatively linked to a gene expression sequence which directs the expression of the antigen nucleic acid within a eukaryotic cell. The gene expression sequence is any regulatory nucleotide sequence, such as a promoter sequence or promoter-enhancer combination, which facilitates the efficient transcription and translation of the antigen nucleic acid to which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, b-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art. In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined antigen nucleic acid. The gene expression sequences optionally include enhancer sequences or upstream activator sequences as desired. Preferred promoters for use in a poxviral vector (see below) include without limitation vaccinia promoters 7.5K, H5R, TK, p28, p11 and K1L, chimeric promoters between early and late poxviral promoters as well as synthetic promoters such as those described in Chakrabarti et al. (1997, Biotechniques 23, 1094-1097), Hammond et al. (1997, J. Virological Methods 66, 135-138) and Kumar and Boyle (1990, Virology 179, 151-158).

The promoter is of special importance and the present invention encompasses the use of constitutive promoters which direct expression of the nucleic acid in many types of host cells and those which direct expression only in certain host cells or in response to specific events or exogenous factors (e.g. by temperature, nutrient additive, hormone or other ligand). Suitable promoters are widely described in literature and one may cite more specifically viral promoters such as RSV, SV40, CMV and MLP promoters. Preferred promoters for use in a poxviral vector include without limitation vaccinia promoters 7.5K, H5R, TK, p28, p11 and K1L, chimeric promoters between early and late poxviral promoters as well as synthetic promoters such as those described in Chakrabarti et al. (1997, Biotechniques 23, 1094-1097), Hammond et al. (1997, J. Virological Methods 66, 135-138) and Kumar and Boyle (1990, Virology 179, 151-158).

Those skilled in the art will appreciate that the regulatory elements controlling the expression of the nucleic acid molecule of the invention may further comprise additional elements for proper initiation, regulation and/or termination of transcription (e.g. polyA transcription termination sequences), mRNA transport (e.g. nuclear localization signal sequences), processing (e.g. splicing signals), and stability (e.g. introns and non-coding 5′ and 3′ sequences), translation (e.g. peptide signal, propeptide, tripartite leader sequences, ribosome binding sites, Shine-Dalgamo sequences, etc.) into the host cell or organism.

Alternatively, the recombinant vector in use in the present invention can further comprise at least one nucleic acid encoding at least one cytokine. Suitable cytokines include without limitation interleukins (e.g. IL-2, IL-7, IL-15, IL-18, IL-21) and interferons (e.g. IFNγ, INFα), with a special preference for interleukin IL-2. When the recombinant vaccine of the invention comprises a cytokine-expressing nucleic acid, said nucleic acid may be carried by the recombinant vector encoding the one or more antigen(s) or by an independent recombinant vector which can be of the same or a different origin.

Infectious viral particles comprising the above-described recombinant viral vector can be produced by routine process. An exemplary process comprises the steps of:
a. introducing the viral vector into a suitable cell line,
b. culturing said cell line under suitable conditions so as to allow the production of said infectious viral particle,
c. recovering the produced infectious viral particle from the culture of said cell line, and
d. optionally purifying said recovered infectious viral particle.

Cells appropriate for propagating adenoviral vectors are for example 293 cells, PERC6 cells, HER96 cells, or cells as disclosed in WO 94/28152, WO 97/00326, U.S. Pat. No. 6,127,175.

Cells appropriate for propagating poxvirus vectors are avian cells, and most preferably primary chicken embryo fibroblasts (CEF) prepared from chicken embryos obtained from fertilized eggs.

The infectious viral particles may be recovered from the culture supernatant or from the cells after lysis (e.g. by chemical means, freezing/thawing, osmotic shock, mecanic shock, sonication and the like). The viral particles can be isolated by consecutive rounds of plaque purification and then purified using the techniques of the art (chromatographic methods, ultracentrifugation on caesium chloride or sucrose gradient).

According to one special embodiment, the Invention relates to a method as above described wherein said human disease is cancer.

According to a preferred embodiment, said cancer is for example breast cancer, colon cancer, kidney cancer, rectal cancer, lung cancer, cancer of the head and neck, renal cancer, malignant melanoma, laryngeal cancer, ovarian cancer, cervical cancer, prostate cancer, non Small cell Lung Cancer.

According to one special embodiment, the Invention relates to a method as above described wherein said human disease is infectious disease.

According to a preferred embodiment, said infectious disease is a viral induced disease, such as for example disease induced by HIV, HCV, HBV, HPV, and the like.

As used herein, the term “low to moderate immune response” is well known from those skilled in the art. More specifically, it means a qualified and/or quantified measure in a given standard immune assay such as index of stimulation in a proliferation assay, a production of cytokines in an ELISA or flow cytometry assay, a CTL assay, an ELISPOT assay, a production of antibodies measured by ELISA, the modulation of expression of cell surface markers evaluated by flow cytometry or microscopy. The said “low to moderate immune response”, or the ability to induce an immune response (e.g. anti-HPV or anti-HCV or anti-MUC1 immune response) upon administration in a patient, can be evaluated either in vitro or in vivo using a variety of assays which are standard in the art. For a general description of techniques available to evaluate the onset and activation of an immune response, see for example Coligan et al. (1992 and 1994, Current Protocols in Immunology ed J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELIspot), by determination of the activation status of immune effector cells (e.g. T cell proliferation assays by a classical [³H] thymidine uptake), by assaying for antigen-specific T lymphocytes in a sensitized subject (e.g. peptide-specific lysis in a cytotoxicity assay). The ability to stimulate a humoral response may be determined by antibody binding and/or competition in binding (see for example Harlow, 1989, Antibodies, Cold Spring Harbor Press). The method of the invention can also be further validated in animal models challenged with an appropriate tumor-inducing agent (e.g. HPV-E6 and E7-expressing TC1 cells) to determine anti-tumor activity, reflecting an induction or an enhancement of an immune response.

According to one special embodiment, said “low to moderate immune response” is an humoral immune response.

According to another special embodiment, said “low to moderate immune response” is a cellular immune response, more particularly a T cell immune response, and even more particularly a CD8+ (Cytotoxic T Lymphocytes) immune response.

According to a preferred embodiment, said “low to moderate immune response” is a specific immune response directed towards at least one antigen, more specifically a tumour-specific or -related antigens and/or viral antigen, and in a special embodiment a said targeted antigen. In special case, said targeted antigen is MUC1.

In a further embodiment there is provided the use of an immunogenic composition comprising all or part of a targeted antigen for the manufacture of a medicament for treating a patient for human disease in a particular patient population wherein the patients of said population have elicited a low to moderate immune response towards said antigen (i.e. the prior immune response) and have not received any prior exogenously supplied pharmaceutical immunogenic composition comprising (i) all or part of the a targeted antigen and/or (ii) at least one recombinant vector encoding the said targeted antigen.

In a further embodiment there is provided the use of an immunogenic composition for the manufacture of a medicament for raising an immune response to a targeted antigen (i.e. the raised immune response) in a patient for treating human disease in a particular patient population wherein the patients of said population have elicited a low to moderate immune response towards an antigen (i.e. the prior immune response) and have not received any prior exogenously supplied pharmaceutical immunogenic composition comprising (i) all or part of the said targeted antigen and/or (ii) at least one recombinant vector encoding the said targeted antigen.

In another embodiment there is provided the use of an immunogenic composition for the manufacture of a medicament for raising an immune response to a targeted antigen (i.e. the raised immune response) in a patient for treating human disease in a particular patient population wherein the patients of said population have elicited a low to moderate immune response towards the said targeted antigen (i.e. the prior immune response) and have not received any prior exogenously supplied pharmaceutical immunogenic composition comprising (i) all or part of the said antigen and/or (ii) at least one recombinant vector encoding the said antigen.

According to one special embodiment, said “raised immune response” and “low to moderate immune response” in said patient population are directed towards a tumour-specific or -related antigens and/or viral antigen. According to one embodiment, said “raised immune response” and “low to moderate immune response” in said patient population are directed towards distinct antigens. According to one special embodiment, said “raised immune response” and “low to moderate immune response” in said patient population are both directed towards MUC1 antigen. According to another special embodiment, said “raised immune response” and “low to moderate immune response” in said patient population are both T cell immune response, and preferably CD8+ (Cytotoxic T Lymphocytes) immune response.

It appears that the patients of the particular patient population identified according to the invention are actually a) able to produce on their own an immune response (e.g. a T cell response) (i.e. prior immune response) to their disease (i.e. towards an antigen associated, directly or indirectly, to their disease (e.g. HPV or MUC1, respectively) and b) that the immunogenic composition subsequently administered to said patient is effectively boosting that prior immune response (i.e. the raised immune response) and/or extending the survival rate of treated patients compared to treated patients who have not elicited a low to moderate immune response towards an antigen as disclosed above.

Thus the present invention further concerns a method for extending the survival rate of a patient treated for human disease by administering an immunogenic composition, said method comprising the following steps:
selection of patient in a patient population composed of patients that have elicited a low to moderate immune response towards to the said targeted antigen and that have not received any prior exogenously supplied pharmaceutical immunogenic composition comprising (i) all or part of the said targeted antigen(s) and/or (ii) at least one recombinant vector encoding the said targeted antigen(s),
administering to said selected patients the said immunogenic composition.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### FIGURE LEGENDS

FIG. 1: Clinical Phase II study TG4010.06 (Kidney cancer). Correlation between MUC1-specific cellular immune response prior to TG4010 immunization and longer patient survival. Dashed line: Patients who had ELISpot evidence for MUC1-specific T cell response before immunization with TG4010. Solid line: patients who had no evidence of MUC1-specific T cell response, by ELISpot, at baseline. Circles represent complete data, where the date of death is known. Crosses represent censored data which indicates patients who were still alive at the time of analysis or who were lost to follow-up.

FIG. 2: Clinical Phase II study TG4010.05 (Lung cancer). Correlation between MUC1-specific cellular immune response prior to TG4010 immunization and longer patient survival. Dashed line: Patients who had ELISpot evidence for MUC1-specific T cell response before immunization with TG4010. Solid line: patients who had no evidence for MUC1-specific T cell response at baseline. Circles represent complete data, where the date of death is known. Crosses represent censored data which indicates patients who were still alive at the time of analysis or who were lost to follow-up. In this study, technical difficulties with the cell transport and freezing resulted in poorer cell condition than the cells used for FIG. 1. For this reason, a level of 5 spots per 10⁵ PBMC, which is 1.5× the background control, was accepted as a positive response to MUC1. The criterion for the positive controls was the same as was used for the data in FIG. 1.

### EXAMPLES

Venous blood samples were taken from metastatic kidney cancer patients who participated in a Phase II clinical study with the immunotherapeutic vaccine TG4010 (MVA vector encoding MUC1 antigen). These patients have never been treated before by administration with MUC1 antigen or nucleic acid encoding it, or any related antigen. Blood samples were drawn prior to TG4010 immunization, and at several time points following the initial TG4010 immunization, while the patient was on the study. Blood samples were collected in Becton Dickenson CPTR® tubes and sent to a central lab for processing to peripheral blood mononuclear cells and freezing in liquid nitrogen. PBMC were then sent, in batch, to another central lab for analysis of MUC1 specific cellular immune response.

PBMC were assessed for MUC1-specific CD8+ (phenotype of cytolytic T cells) responses by ELISpot (Enzyme Linked Immuno spot) assay using the Diaclone (Besoncon, France) system. ELISpot is a very well known assay used to assess CD8+ T cell responses to an antigen. Briefly: PBMC are put into ELISpot assay plates in culture medium alone or together with and antigen, in this case, a peptide from the MUC1 sequence. The MUC1 peptides used are specific for the HLA type of the patient. PBMC are also assessed for ELISpot response to positive control peptides from common viral proteins. This ensures that the assay is working and that the cells are in a fit state to respond in the ELISpot assay. Cells are incubated for 16 hours in the ELISpot plates. ELISpot plate wells are coated with a capture antibody which is specific for human Interferon gamma. That way, when cells are washed out of the plate following the 16 hour culture period, the area surrounding a cell which produces interferon gamma in response to the peptide, will have the interferon attached to the bottom of the well. A second interferon gamma antibody, coupled to an enzyme is then added and after washing the enzyme substrate added. The result is a coloured spot wherever there was a T cell which reacts with the peptide. The spots are then counted and the count is the number of T cells in the suspension which are specific for that peptide. All assays are done in triplicate.

MUC1 is a self antigen and responses were expected to be weak. Therefore the specific T cells were first expanded in tissue culture with a 6 day in an in vitro sensitization step prior to the ELISpot assay. During this time, patient PBMC are cultured with short synthetic peptides from the human MUC1 sequence which have been identified to bind to the HLA haplotypes (EP 1210430), and interleukin-2 (IL2) to aid T cell proliferation. After the culture period, sensitized lymphocytes are washed and assessed for specific T cell activity in the ELISpot assay as described above, using the same peptides or no peptides at all as the background control.

In the ELISpot assay, a response is considered positive if there are at least 10 spots per well (10⁵ PBMC) greater than in the background control wells and if that number of spots represents at least 1.5× the number of spots in the wells containing the background control (with no peptides). In addition, response to MUC1 is considered positive only if the positive control is positive, but the same criteria. Similarly, a response to MUC1 (fewer than 10 spots per well) is considered negative only if the response to the positive controls is positive.

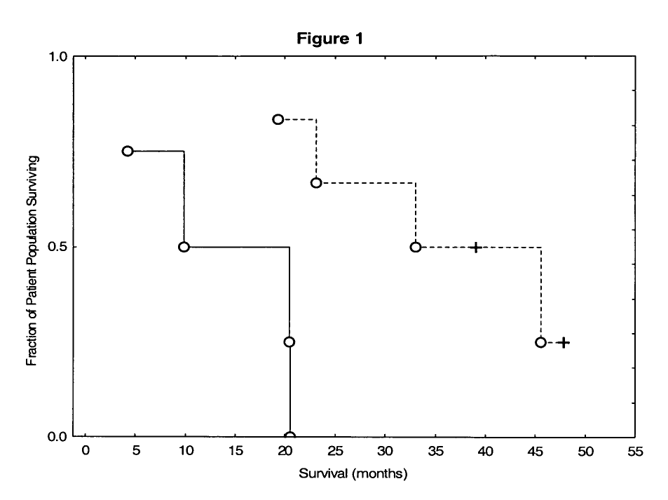

PBMC from patients in the kidney cancer TG4010 immunotherapy study were tested for MUC1-specific CD8+ cellular immune response by ELISpot. PBMC from patients taken prior to TG4010 were tested, as were PBMC taken from patient following TG4010 immunizations. There was a significant correlation between patients who had MUC1-specific ELISpot response prior to immunization with TG4010 and extended patient survival (FIG. 1) when compared to patient PBMC which were evaluable for MUC1-specific ELISpot response but had no such MUC1-specific response at baseline. In addition, all patients who had MUC1-specific ELISpot response at baseline (i.e. prior immunisation with the immunogenic composition TG4010) enjoyed an overall survival which was greater than the median survival of 19 months, calculated on the basis of all 37 patients enrolled in the study.

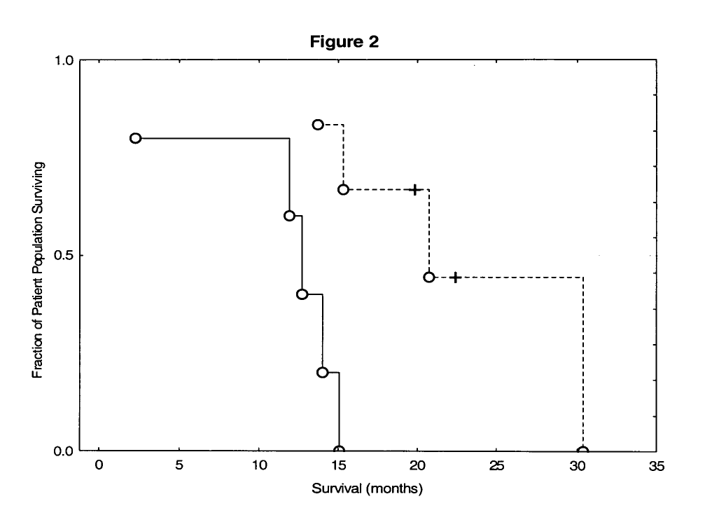

The data shown in FIG. 1 is supported from data from a similar Phase II clinical study with TG4010 in patients with advanced Non-small cell lung cancer (FIG. 2). Thirty one patients were evaluable for MUC1-specific ELISpot and 29 patients were evaluable for MUC1-specific ELISpot responses at baseline. Eleven patient responses met the positive control criteria. Only these data are shown in FIG. 2. Similar to the data shown in FIG. 1, lung cancer patients who showed detectable MUC1-specific ELISpot responses by PBMC from blood taken at baseline (i.e. patients that have elicited a low to moderate immune response towards to the said targeted antigen, MUC1, and that have not received any prior exogenously supplied pharmaceutical immunogenic composition comprising (i) all or part of the said targeted antigen and/or (ii) at least one recombinant vector encoding the said targeted antigen) prior to any immunization with TG4010, survived significantly longer than patients who showed no detectable MUC1-specific ELISpot responses by PBMC from blood taken at baseline. In addition, all patients with a MUC1-specific T cell response, as measured by ELISpot, had a longer survival than the median survival of all 65 patients in the suty. Median Survival was 13.7 months.

1.-9. (canceled)

10. A method for extending the survival rate of a patient treated for human disease by administering an immunogenic composition comprising (i) all or part of one targeted antigen and/or (ii) at least one recombinant vector encoding one targeted antigen, said method comprising the following steps:
selecting a patient from a patient population of patients that have elicited a low to moderate immune response towards said targeted antigen and that have not received any prior exogenously supplied pharmaceutical immunogenic composition comprising (i) all or part of the said targeted antigen(s) and/or (ii) at least one recombinant vector encoding the said targeted antigen(s),
administering to said selected patient the said immunogenic composition.

11. The method as defined by claim 10, wherein said immunogenic composition administered to said patient further comprises (iii) at least one immune response modifier.

12. The method as defined by claim 10, wherein said human disease is cancer.

13. The method as defined by claim 12, wherein said cancer comprises Non Small Cell Lung Cancer or kidney cancer.

14. The method as defined by claim 10, wherein said targeted antigen comprises a tumour specific antigen.

15. The method as defined by claim 14, wherein said targeted antigen comprises a MUCI1.

16. The method as defined by claim 10, wherein said low to moderate immune response comprises a humoral immune response.

17. The method as defined by claim 10, wherein said low to moderate immune response comprises a cellular immune response.

18. The method as defined by claim 17, wherein said immune response comprises a CD8+ immune response.

19. An immunogenic composition comprising (i) all or part of one targeted antigen and/or (ii) at least one recombinant vector encoding one targeted antigen, formulated into a medicament for extending the survival rate of a particular patient population.

20. The immunogenic composition as defined by claim 19, comprising (i) all or part of one targeted antigen and/or (ii) at least one recombinant vector encoding one targeted antigen and/or (iii) at least one immune response modifier.

21. The immunogenic composition as defined by claim 19, wherein said targeted antigen is a tumour specific antigen.

22. The immunogenic composition as defined by claim 21, wherein said targeted antigen is a MUC1.

23. The immunogenic composition as defined by claim 19, said patient population exhibiting low to moderate immune response comprises a humoral immune response.

24. The immunogenic composition as defined by claim 23, wherein said low to moderate immune response comprises a cellular immune response.

25. The immunogenic composition as defined by claim 24, wherein said immune response comprises a CD8+ immune response.
